(19) United States
(12) Patent Application Publication
(10) Pub. No.: US 2010/0030269 A1
(43) Pub. Date: Feb. 4, 2010

(54) INTERSPINOUS SPINAL PROSTHESIS

(76) Inventor: Jean Taylor, Cannes (FR)

Correspondence Address: MEDTRONIC;Attn: Noreen Johnson - IP Legal Department, 2600 Sofamor Danek Drive, Memphis, TN, 38132, US

(21) Appl. No.: 12/439,843

(22) PCT Filed: Sep. 5, 2007
(86) PCT No.: PCT/IB07/02566

§ 371 Date: Mar. 17, 2009

Related U.S. Application Data
(60) Provisional application No. 60/842,652, filed on Sep. 7, 2006.

(30) Foreign Application Priority Data
Oct. 20, 2006 (FR) 06 09209

(51) Int. Cl. A61B 17/70

(52) U.S. Cl. 606/248

(57) ABSTRACT

This spinal prosthesis (2) comprises:—a U-shaped implant (3), comprising an intermediate portion (5) and two side portions (6), at least the intermediate portion (5) being shock absorbing; this implant (3) is intended to envelope the spinous process of a first vertebra (L5), said intermediate portion (5) being intended to be engaged between this spinous process and an area of a second vertebra (S1), underlying; and—a link (4) crossing through the intermediate portion (5) and forming two side strands which are able to go along-side or cross said side portions (6) and to be connected to each other so as to closely stick said intermediate portion (5) against the lower edge of the spinous process of said first vertebra (L5).

The present invention relates to an interspinous spinal prosthesis.

### STATE OF THE ART

Interspinous spinal prostheses have been developed as an alternative to interpedicular osteosynthesis devices. The interspinous gap, thus maintained, provides a certain degree of stability by reducing the play between the posterior rims of the vertebrae.

It is essential for this type of implant to conform to the morphology of the spinous processes to prevent migration or forward or rear sliding. This requirement is, however, problematic between the fifth lumbar vertebra (L5) and the first sacral vertebra (S1) because the S1 spinous process very often protrudes very little (agenesis) and therefore cannot constitute a stable bearing point.

The solutions imagined to date have tried to use bearing on the sacrum, either through hooks incorporated in the device and bearing on the upper edge of the sacrum, or using side attachments or transverse links fastened by screws going through the ala sacralis. Due to the specific biomechanics of the lumbo-sacral joint, all of these solutions are both delicate to implant and unsatisfactory from a mechanical standpoint. However, degenerative pathologies affect several consecutive vertebral joints or evolve such that they affect several consecutive vertebral joints. It is therefore essential to be able to extend the treatment method from a given vertebra toward the joint located above and especially the joint located below, namely L5-S1 involving an L4-L5 treatment.

Documents FR 2 623 085, WO 2005/110258, WO 2006/086241 and FR 2 828 398 describe various types of interspinous implants, but which do not provide a satisfactory solution to the aforementioned problem of providing an implant for which migration or forward or rear sliding is prevented, which remaining relatively easy to implant and providing satisfaction from a mechanical standpoint.

### SUMMARY OF THE INVENTION

To resolve the abovementioned problem, the prosthesis according to the present invention comprises:
a U-shaped implant, comprising an intermediate portion and two side portions, at least the intermediate portion being shock absorbing; this implant is intended to envelope the spinous process of a first vertebra (generally L5), said intermediate portion being intended to be engaged between this spinous process and an area (generally the top of the posterior rim) of a second vertebra (generally S1), underlying; and
a link crossing through the intermediate portion and forming two side strands which are able to go alongside or cross (transfix) said side portions and to be connected to each other so as to closely stick sad intermediate portion against the lower edge of the spinous process of said first vertebra (L5).

The invention was designed based on the two following observations, relative to the L5-S1 stage: on one hand, the supraspinous ligamentary continuity ends at the L5 vertebra, then irradiates; on the other hand, the axis of the spinal cord forms a closed angle, meaning acute, with the lower edge of the spinous process of said first vertebra L5. As a result, preserving the supraspinous ligamentary continuity is not imperative at this level, whereas the support provided by the spinal process of said first vertebra L5 is anatomically weak. During an extension of the vertebral column, the lower edge of the spinal process of the L5 vertebra will come into contact, and, if necessary, bear against the spur formed by the spinal process of said second vertebra, namely the S1 vertebra in this case, which is triangular in shape. The implant according to the invention, slid under this spinal process, will be stuck in the interspinous space while maintaining a gap favorable to soothing the painful newly formed interspinous and inter-joint contacts, while also stretching the capsuloligamentary elements.

In a first embodiment, the implant is distinct and separate from an interspinous implant intended to be placed at the vertebral joint situated above said first vertebra (generally L5), namely between said first vertebra (L5) and a third vertebra (L4). In this case, the implant is U-shaped and is provided with a link at its edge or going through it. The implant is forcibly slid into the interspinous space of the L5 and S1 vertebrae and fits under the lower edge of the L5 spinous process, against which it bears. The link keeps the implant in place by squeezing.

In a second embodiment, the implant is integral with a shock absorbing upper interspinous part intended to be provided on the vertebral joint between said first vertebra (L5) and a third vertebra (L4) above first vertebra (L5); the implant defines, with this upper interspinous part, an opening allowing close, even more or less forced, insertion of the implant on the spinous process of said first vertebra (generally L5). In this case, the link goes through said upper interspinous part to allow the joining of its two side strands.

The implant according to the invention can be “for extension”, meaning lengthening, having the effect of increased maintaining of the interspinous device (which is separate from the implant in the first embodiment) or, in said second embodiment, of said upper interspinous part, by preventing forward or rear sliding of its intermediate part.

The implant according to the invention, thanks to its presence on the underlying vertebral joint (generally L5-S1) and its perfect fixing to the spinous process of said first vertebra (L5), solves the problem of sliding of an interspinous prosthesis being provided on the vertebral joint between said first and third vertebrae (generally L4-L5) relative to the upper edge of the spinous process of said first vertebra (L5), this risk of sliding resulting from the tilt of this upper edge.

All of the preceding remarks may be transposed from the lumbar area to the cervical and dorsal area.

The implant is preferably made of a viscoelastic material, particularly with a fairly high gradient, optimizing both the stability of the implant by a self-tightening effect around the spinous process and providing a shock absorbing effect for the spinous processes of the first and second vertebrae, generally L5 and S1, respectively. Thus, one can compensate for agenesis of the spinous process of said second vertebra (S1) given that the bearing is previously moved to the posterior junction of the two half-laminae of the sacrum.

The implant may comprise a core and an outer covering.

The core may be a composite structure. In particular, the core may comprise an inner element made of a non-compressible material and an outer element covering the inner element, forming a thicker layer in a malleable viscoelastic shock absorbing material.

The elasticity module of this malleable viscoelastic shock absorbing material is advantageously less than that of a cortical bone so as to avoid bone weakening.

The outer covering may be in a woven textile material or in any other material with a suitable canvas.

Said intermediate portion of the implant may comprise at least one recess or notch intended to allow precise application of the implant against the lower edge of the spinous process in said first vertebra (generally L5) and/or against the second vertebra (generally S1), and therefore allowing stabilization of this implant relative to that or these spinous process(es).

Preferably, the free end of at least one of the side portions of the implant comprises a recess or a notch enabling the implant to interlock with an interspinous implant intended to be placed between said first and third vertebrae. Preferably, each of the free ends of the two side portions of the implant comprises a recess or notch of this type.

Moreover, the materials required for each segment of this assembly thus formed may differ, thereby forming a composite assembly with variable stiffness.

### BRIEF DESCRIPTION OF THE FIGURES

The figures illustrate two embodiments of the invention.

FIG. 1 is a back view, after implantation, according to a first embodiment;

FIG. 2 is a view similar to FIG. 1, according to a variation of the embodiment, and

FIG. 3 is a back view, after implantation, according to a second embodiment.

### DISCUSSION OF PREFERRED EMBODIMENTS

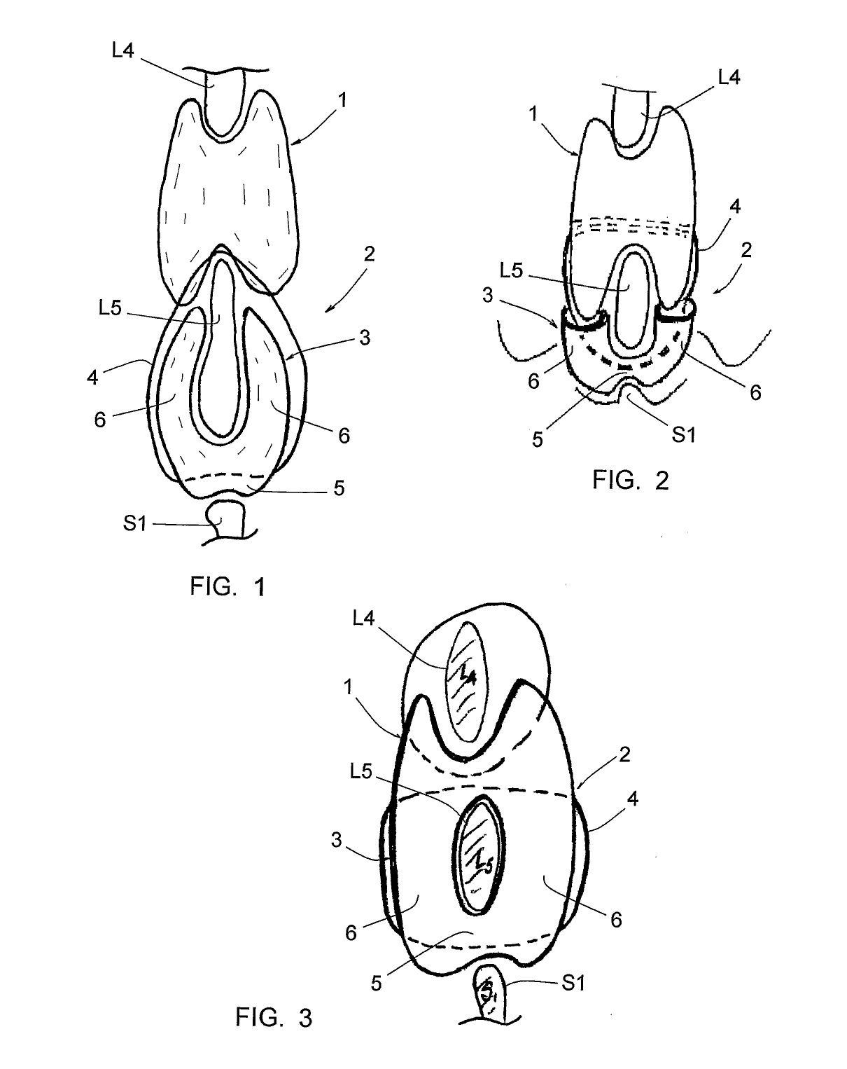

FIG. 1, annexed, shows three successive spinous processes, generally L4, L5 and S1. Between the L4 and L5 spinous processes is placed an interspinous implant 1, in particular that known under the name “DIAM”.

The prosthesis 2 according to the invention comprises an implant 3 and a link 4.

The implant 3 has a U-shape, meaning it comprises an intermediate portion 5 and two side portions 6. At least the intermediate portion 5 is shock absorbing. This implant 3 is intended to envelope the spinous process of L5, said intermediate portion 5 being intended to be engaged between this spinous process and the S1 spinous process.

The link 4 goes through said intermediate portion 5 and forms two side strands which can go alongside said side portions 6 and be connected to each other so as to closely stick said intermediate portion 5 against the lower edge of the L5 spinous process.

FIG. 2 shows a variation of this embodiment wherein the free ends of the side portions 6 comprise recesses enabling them to interlock on the free ends of the lower protruding side portions comprised by the interspinous implant 1. The link 4 is then engaged through the central part of the implant 1.

In a second embodiment, shown in FIG. 3, the implant 3 is integral with an upper shock absorbing interspinous part 1 comparable to the interspinous implant 1 of the preceding version. The implant 3 defines, with this upper interspinous part 1, an opening allowing close insertion on the spinous process of the L5 vertebra. In this case, the link 4 goes through said upper interspinous part 1 to enable the connection of its two side strands.

1.-9. (canceled)

10. A spinal prosthesis comprising:
a U-shaped implant configured to envelope a first spinous process; the implant comprising:
an intermediate portion and two side portions;
at least the intermediate portion being shock absorbing;
the intermediate portion configured to be engaged between the first spinous process and an adjacent second lower vertebrae;
a link crossing though the intermediate portion and forming two side strands are able to go alongside or cross said side portions and to be connected to each other so as to closely stick said intermediate portion against a lower edge of the vertebrae associated with the first spinous process.

11. The spinal prosthesis of claim 10 further comprising a second implant, distinct from the first implant; the second implant configured to be placed at a vertebral joint between the vertebrae associated with the first spinous process and an superiorly adjacent third vertebrae.

12. The spinal prosthesis of claim 10 wherein the implant further comprises a shock absorbing upper part configured to be disposed at a vertebral joint between the vertebrae associated with the first spinous process and an superiorly adjacent third vertebrae; wherein the implant further comprises an opening for receiving the first spinous process.

13. The spinal prosthesis of claim 10 wherein the implant is made of a viscoelastic material.

14. The spinal prosthesis of claim 10 wherein the implant comprises a core and an outer covering.

15. The spinal prosthesis of claim 14 wherein the core is a composite structure comprising an inner element made of a non-compressible material and an outer element covering the inner element; the outer element forming a malleable viscoelastic shock absorbing layer.

16. The spinal prosthesis of claim 14 wherein the outer covering comprises a woven textile material.

17. The spinal prosthesis of claim 10 wherein the intermediate portion comprises at least one recess configured to receive one of the lower edge of the vertebrae associated with the first spinous process or the second vertebrae to help stabilize the implant relative thereto.

18. The spinal prosthesis of claim 10 wherein a free end of at least one of the side portions comprises a recess configured to enable the implant to interlock with an second implant, the second implant configured to be placed at a vertebral joint between the vertebrae associated with the first spinous process and an superiorly adjacent third vertebrae.

19. A spinal implant comprising:
a U-shaped implant body having first and second arms and an intermediate section disposed therebetween; the intermediate section being elastically deformable and comprising a lateral passage extending from a first surface opening proximate the first arm to a second surface opening proximate the second arm; the first and second arms having free ends distal from the intermediate section terminating at tips;
a tether extending through the lateral passage and having a length greater than a combined length of the implant body so as to extend beyond the tips of the first and second arms;
wherein, when the implant body is placed between the L5 and S1 vertebrae, with the spinous process of the L5 vertebrae is disposed between the first and second arms and the intermediate section extending through a sagittal plane defined by the L5 and S1 vertebrae, and with the tether extending above the superior surface of the spinous process of the L5 vertebrae:
the intermediate section is configured so as to be disposed between the inferior edge of the spinous process and the S1 vertebrae, with the intermediate section in contact with the inferior surface of the spinous process of the L5 vertebrae, and
the tether extends along an exterior surface of the arms so as to press the arms against the spinous process of the L5 vertebrae.

20. The spinal implant of claim 19 wherein the tips of the first and second arm include recesses oriented away from the intermediate section.

21. The spinal implant of claim 19 wherein the first arm comprises the first surface opening and the second arm comprises the second surface opening.

22. The spinal implant of claim 19 wherein the intermediate section comprises a viscoelastic material.

23. The spinal implant of claim 19 wherein the intermediate section comprises a recess opening away from the arms and configured to receive a portion of the S1 vertebrae.
